(19) United States
(12) Patent Application Publication
(10) Pub. No.: US 2026/0360465 A1
(43) Pub. Date: Nov. 12, 2026

(54) METHOD TO EVALUATE THE CONSPICUOUSNESS OF AN EPITOPE TOWARDS THE REPERTOIRE OF T-CELL RECEPTORS

(71) Applicant: IMMUNEWATCH BV, Antwerpen (BE)

(72) Inventors: Pieter MEYSMAN, Antwerpen (BE); Max VAN HOUCKE, Antwerpen (BE)

(21) Appl. No.: 19/133,136

(22) PCT Filed: Nov. 30, 2023
(86) PCT No.: PCT/EP2023/083687

§ 371 Date: May 27, 2025

Related U.S. Application Data
(60) Provisional application No. 63/429,296, filed on Dec. 1, 2022.

(51) Int. Cl. G16B 20/00; G01N 33/50; G16B 15/30; G16B 30/00; G16B 40/20

(52) U.S. Cl. CPC G16B 20/00; G01N 33/505; G16B 15/30; G16B 30/00; G16B 40/20; G01N 2500/10

(57) ABSTRACT

The current invention relates to a method of predicting T-cell response to a query epitope with a known epitope-TCR binding comprising calculating conspicuousness score of said epitope based on the number of known TCR sequences or TCR clusters responsive to said epitopes or by a centrality metric for each said epitopes in the epitope-TCR graph. The invention further relates to a second method to predict T-Cell response of any arbitrary epitopes using a machine learning algorithm. The T-cell response of epitopes can be used for the selection of molecules for vaccine and/or non-immunogenic compositions. The invention further relates to a third method for producing vaccine, to a vaccine, a biological and a data base.

### FIELD OF THE INVENTION

The present invention is in the field of immunology and medicine. The present invention is particularly in the field of prediction of immunogenicity of a peptide.

### BACKGROUND

Developing a new vaccine or other T-cell-based therapy aims to elicit an adaptive immune response for which T-cells are one of the most important drivers. The epitopes or antigens included in the therapy are selected based on their ability to elicit this response, which is termed immunogenicity.

Inversely, the development of certain products such as therapeutic biologicals aims to prevent any immune response against the product as this may cause the degradation of the product, making the therapy inefficient. In such situations, a T-cell response is not desired, and the product should be of low immunogenicity.

The current state of the art uses major histocompatibility complex (MHC) binding as a proxy for T-cell immunogenicity. U.S. Pat. No. 11,183,272, EP2550529, WO2019006022, US20210104294 disclose several methods to predict whether an epitope can be coupled and consequently presented by an MHC molecule as the presentation of the epitope by an MHC molecule is considered to be the most important step for T-cell activation and consequently, for elicitation of an adaptive immune response. The prediction of immunogenicity by using only MHC-binding approach considers one aspect of T-cell activation and neglects the other features that can also be used as indicators of T-cell response.

The T-cell receptor (TCR) is a protein complex found on the surface of T cells, or T lymphocytes that is responsible for recognizing fragments of antigen as peptides bound to major histocompatibility complex (MHC) molecules. CN111429965 and WO2022072722 disclose other methods that attempt to predict if a single epitope can be bound by a specific T-cell receptor (TCR). However, these methods require prior knowledge of TCR sequences, which is not known in many applications. In addition, the prediction of this binding is highly inaccurate, and most reported performances in this field are due to imbalances or biases in the test set.

US20210147929 discloses a method to address the low accuracy prediction by acquiring an epitope-specific training data set, however, this is only possible for a limited number of epitopes.

There remains a need in the art for an improved method for more precise prediction of the immunogenicity of any potential epitope.

### SUMMARY OF THE INVENTION

The present invention and embodiments thereof serve to provide a solution to one or more of above-mentioned disadvantages. To this end, in a first aspect the present invention relates to a method of predicting T-cell response to a query epitope with a known epitope-TCR binding according to claim 1. The method comprises calculating conspicuousness score of said epitope based on the number of known TCR sequences or TCR clusters responsive to said epitopes or by a centrality metric for said query epitope in the epitope-TCR bipartite graph wherein said calculated conspicuousness score represents the T-cell response to said query epitope.

In a second aspect, the present invention relates to a method of predicting T-cell response to a candidate epitope according to claim 6. The method comprises generating a training data set by calculating a conspicuousness score of a plurality of epitopes with known epitope-TCR binding wherein said score is calculated based on the number of known TCR sequences responsive to each said epitopes or number of TCR clusters, grouped by TCR sequence patterns, responsive to each said epitope or by a centrality metric for each said epitopes in the epitope-TCR bipartite graph; calculating conspicuousness score of said epitope using a machine learning algorithm model trained using said training data set to predict T-cell response of said epitope. A specific preferred embodiment relates to an invention according to claim 7.

In a third aspect, the present invention relates to a method of producing a vaccine composition according to claim 20.

In a fourth aspect the present invention relates to a vaccine composition according to claim 22.

In a fifth aspect, the present invention relates to a non-immunogenic composition according to claim 23.

In a sixth aspect, the present invention relates to a data set according to claim 24.

### DESCRIPTION OF FIGURES

The following description of the figures of specific embodiments of the invention is merely exemplary in nature and is not intended to limit the present teachings, their application or uses.

FIG. 1 schematically presents a conspicuousness score calculation and training of the machine learning model.

FIG. 2 shows the Receiver Operator Characteristic for the independent test data.

### DETAILED DESCRIPTION OF THE INVENTION

The present invention concerns a method for predicting the T-cell response of an epitope by its conspicuousness to a repertoire of TCRs. The method is distinct as it focuses on the TCR diversity of the T-cell response after MHC binding has been achieved that is novel in the view of prior art. The methods disclosed can further predict the T-cell response of an arbitrary epitope without any known epitope-TCR binding via calculating their conspicuousness to a repertoire of TCRs by using machine learning algorithms.

### DEFINITIONS

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

As used herein, the terms “protein,” “polypeptide,” and “peptide” refer to a molecule comprising amino acids joined via peptide bonds. In general, “peptide” is used to refer to a sequence of 40 or less amino acids and “polypeptide” is used to refer to a sequence of greater than 40 amino acids.

As used herein, the term, “synthetic polypeptide,” “synthetic peptide” and “synthetic protein” refer to peptides, polypeptides, and proteins that are produced by a recombinant process (i.e., expression of exogenous nucleic acid encoding the peptide, polypeptide or protein in an organism, host cell, or cell-free system) or by chemical synthesis.

As used herein, the term “immunogen” refers to a molecule which stimulates a response from the adaptive immune system, such as a T-cell response. The unlimiting examples of said responses may comprise an antibody response, a cytotoxic T-cell response, a T helper response, and a T-cell memory. An immunogen may stimulate an upregulation of the immune response with a resultant inflammatory response or may result in down-regulation or immunosuppression. Thus, the T-cell response may be a T regulatory response. An immunogen also may stimulate a B-cell response and lead to an increase in antibody titer. Another term used herein to describe a molecule or combination of molecules which stimulate an immune response is “antigen”.

As used herein the term “epitope” refers to a peptide sequence which elicits an immune response, from either T cells or B cells or antibody. An epitope may be a linear peptide or may comprise several discontinuous sequences which together are folded to form a structural epitope. Typically, T-cell epitopes are presented bound to a MHC molecule on the surface of an antigen-presenting cell.

As used herein, the term “query epitope” or “candidate epitope” refers to a polypeptide sequence that is predicted to bind to a major histocompatibility protein molecule by computerized methods, or as determined experimentally. As used herein, the term “major histocompatibility complex (MHC)” refers to the MHC Class I and MHC Class II genes and the proteins encoded thereby. Molecules of the MHC bind small peptides and present them on the surface of cells for recognition by T-cell receptor-bearing T-cells.

The T-cell receptor (TCR) is a protein complex found on the surface of T cells, or T lymphocytes that is responsible for recognizing fragments of antigen as peptides bound to MHC molecules. The binding between TCR and antigen peptides is of relatively low affinity and is degenerate: that is, many TCRs recognize the same antigen peptide and many antigen peptides are recognized by the same TCR.

As used herein, the term “conspicuous” epitope or “conspicuousness” of an epitope refers to how visible that epitope is to a set of TCRs or how recognizable the epitope is by a set of TCRs. “Conspicuousness score” of an epitope as used herein refers to a calculated value of the probability of that epitope being visible by a set of TCRs. As such, conspicuousness score represents the conspicuousness of an epitope towards the repertoire of TCRs, effectively approximating the breadth of the T-cell response to an epitope.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

### DETAILED DESCRIPTION

In a first aspect, the invention relates to a method of predicting the T-cell response to a query epitope with known epitope-TCR binding. The method comprises, calculating conspicuousness score of said epitope wherein said score is calculated based on the number of known TCR sequences responsive to said epitope or number of TCR clusters, grouped by TCR sequence patterns, responsive to said epitope or by a centrality metric for each said epitopes in the epitope-TCR graph; wherein said calculated conspicuousness score represents and/or is used to predict the T-cell response to said query epitope.

In embodiments of discloses method, conspicuousness score is calculated by a centrality metric for each said epitopes in the epitope-TCR graph wherein said epitope-TCR graph is a bipartite graph.

In embodiments of the disclosed method, the ways of calculating the conspicuousness score for the epitopes which are present in a database of known epitope-TCR binding pairs are established.

In embodiments, calculated conspicuousness score represents the conspicuousness of an epitope towards the repertoire of TCRs, effectively approximating the breadth of the T-cell response to an epitope. Two main factors can influence the breadth of response: the degeneracy of the TCR sequence that can result in binding and the different possible binding modes that the epitope-TCR complex can exist in. As known to a skilled person in the art, degeneracy is an important feature of the immune response mechanism that permits effective T-cell responses to a vast number of potential peptide sequences complexed to MHC molecules with specificity sufficient to distinguish between self and foreign peptides and thus to avoid autoimmune disease.

In embodiments, the conspicuousness score of an epitope can be calculated from different size of epitope-TCR databases. In embodiments, the higher limit of database of known epitope-TCR binding pairs is unlimited. In further embodiments, said databases may have larger than 75 unique pairs, larger than 100 unique pairs, larger than 1000, larger than 10000, larger than 100000, larger than 1000000, larger than 10000000 (10 million) unique pairs. In preferred embodiments, the data bases have between 100 and 10000000 unique pairs, between 1000 and 10000000, between 10000 and 10000000, between 100000 and 10000000, between 100000 and 1000000, and all the ranges and subranges therein between. According to embodiments, the conspicuousness score can be calculated for any epitope present in the database.

In embodiments, the conspicuousness score is calculated based on the number of known TCRs that are responsive to an epitope for each epitopes present in a database. In further embodiments, the conspicuousness score of an epitope can also be calculated based on the number of TCR clusters that are grouped by TCR sequence patterns and are known to interact with said epitope.

In other embodiments, alternative ways of calculating of the conspicuousness score of an epitope can be achieved by a centrality metric for the epitope in the epitope-TCR bipartite graph, where epitopes belong to one class of nodes, TCRs belong to the other class of nodes, and edges represent known or predicted epitope-TCR associations.

In embodiments, the centrality metric includes, but is not limited to degree centrality, eccentricity centrality, or PageRank centrality.

In further embodiments, one or more transformation is applied to said epitope-TCR bipartite graph to improve the quality of the model and consequently the metric. These transformations include but are not limited to node filtering or edge weighting by measures including our unique confidence or specificity measures for known epitope-TCR pairs.

In preferred embodiments, further transformations are applied to said centrally metric to improve the quality of the metric, preferably said transformations are node filtering or edge weighting.

In embodiments, query epitopes with a conspicuousness score above a predefined threshold are selected for vaccine testing. Examples of suitable thresholds, calculated by using the disclosed method, can be but are not limited to in a range of 0.5 to 100 of raw score. For example, 0.5 to 1, 5 to 100, 10 to 100, 15 to 100, 20 to 100, 30 to 100, 40 to 100, 50 to 100, 60 to 100, 70 to 100, 80 to 100, 85 to 100, 90 to 100, 95 to 100 or 0.5 to 90, 0.5 to 80, 0.5 to 70, 0.5 to 60, 0.5 to 50, 0.5 to 40, 0.5 to 30, 0.5 to 20, 0.5 to 15, 0.5 to 10, 0.5 to 5 of calculated raw value and all the ranges and subranges therein between. In embodiments, suitable threshold values may have Bayesian Poisson Regression (BPR) or P-values in the range of 0.1 to 0.00001, for example 0.05 to 0.00001, 0.01 to 0.00001, 0.001 to 0.00001 and all ranges and subranges therein between. One skilled in the art will appreciate that different threshold values may be chosen in different circumstances for example, the threshold value may vary depending on available candidate epitopes, severity of the indication and/or urgency of the need for a vaccine.

In embodiments, the T-cell response of query epitopes with a conspicuousness score above a predefined threshold is measured and/or confirmed by means of biological assay. Any biological assays used to assess the T-cell response to an epitope, antibody or any other T-cell stimulatory molecules are known in the art can be used to measure/confirm the T-cell response of query epitopes. It is understood that a skilled person will choose suitable biological assays for the assessment of T-cell response. Some non-limiting examples of known methods of measuring T-cell activation include e but is not limited to the assessment of cell proliferation, the assessment of the regulation of activation markers, production of effector cytokines.

In embodiments, the method is a computer-implemented method.

In a second aspect, the present invention relates to a computational method of predicting a T-cell response to a candidate epitope.

In embodiments, the calculation of the conspicuousness score is extended to any arbitrary epitope including epitopes that are not in a database.

In embodiments the computational method comprises, generating a training data set by calculating the conspicuousness score of a plurality of epitopes with known epitope-TCR binding wherein said score is calculated based on the number of known TCR sequences responsive to each said epitopes or number of TCR clusters, grouped by TCR sequence patterns, responsive to each said epitope or by a centrality metric for each said epitopes in the epitope-TCR graph, preferably in the epitope-TCR bipartite graph; predicting conspicuousness score of candidate epitope using a machine learning algorithm model trained using said training data set.

In an embodiment, calculating the conspicuousness score of a candidate epitope comprises the steps of
training the algorithm with training data set;
feeding the algorithm with an amino acid sequence of said query epitope;
conversion of said sequence into numeric features wherein said numerical features represent the physicochemical properties of the epitope such as, sequence length, HLA preference, amino acid frequencies, and molecular properties;
predicting the conspicuousness score of the query epitope using said numeric features.

In an embodiment, predicting the conspicuousness score of a candidate epitope comprises the steps of
training the algorithm with training data set;
feeding the algorithm with an amino acid sequence of said query epitope;
conversion of said sequence into numeric features wherein said numerical features represent the physicochemical properties of the epitope such as, sequence length, HLA preference, amino acid frequencies, and molecular properties;
predicting the conspicuousness score of the query epitope using said numeric features.

In embodiments, said method uses a predictive model, said predictive model is a machine learning model trained using a training dataset.

According to embodiments, the training data set comprises a list of epitopes with known epitope-TCR binding and their conspicuousness score.

The query and candidate epitopes represent a putative minimum amino acid sequence that can be recognized by an immune system component (e.g. by a T-cell receptor). The epitope is preferably a linear epitope.

In embodiments, the input of the algorithm is any amino acid sequence as a candidate epitope. In a preferred embodiment, the amino acid sequence is 5 to 35 amino acids in length such as 5 to 30 amino acids, 5 to 25 amino acids, 5 to 20 amino acids, 5 to 15 amino acids, 5 to 10 amino acids, 5 to 7 amino acids and all the ranges and subranges therein between.

In embodiments, amino acid sequence may be derived from one or more biological or synthetic sources, such as peptides or synthetic peptides. The epitope can be an amino acid sequence translated from a nucleic acid sequence.

In embodiments, the input amino acid sequence is converted into numeric features. These numerical features represent the physicochemical properties of the epitope.

In embodiments, the physicochemical properties of the epitope include but are not limited to the length of the amino acid sequence, HLA preference, amino acid frequencies, and molecular properties.

In embodiments, the output of the algorithm is a single score and said score is a continuous value that represents the conspicuousness of the candidate epitope.

In further embodiments, the conspicuousness score is supplemented by an additional interpretability measure which is preferably generated through a transformation function.

In embodiments, the transformation function includes, but is not limited to the log of the probability (p-value) or the expect value (E-value) where P-value present the probability that the given conspicuousness score is obtained by chance and E-value presents the number of hits to be expected by chance. In embodiments, both p-value and E-value are generated by the background distribution of a set of random epitope sequences.

In embodiments, the operation of the algorithm is based on a machine learning framework which can be any machine learning framework that can translate the numerical input features into a single score.

In embodiments, the machine learning framework is chosen from the non-limiting list of multivariate regression, Bayesian regression, support vector machines, regression forests, linear regression, gradient descent methods, gradient boosting methods and/or neural networks or any combination therein.

In embodiments, weights of the machine learning model are fit on training data. In embodiments, the training data set comprises a list of epitopes and their known conspicuousness score, derived from a TCR-epitope database using the disclosed method.

In embodiments, the conspicuousness score of an epitope is used to predict T-Cell response of said epitope. A low conspicuousness score relates to a narrow or no T-Cell response whereas a high conspicuousness score relates to a broad T-cell response. According to embodiments of present disclosure, when deploying disclosed method for vaccine design, the epitopes with the highest scores should be retained.

The conspicuousness score of an epitope may also be used as a stand-in for B-cell response of the epitope. In embodies, a high conspicuousness score relates to a good antibody response. For example, a high conspicuousness score can relate to an antibody response with a high (e.g. >10 IU) antigen-specific antibody titer after 7 days to 60 days after administration of the epitope or the antigen containing the epitope to an individual or a population.

In embodiments, epitopes with low conspicuousness scores are expected to receive low or no immune response. When deploying this method for prioritizing biologicals according to the lowest probability of an unwanted immune response, the epitopes with the lowest scores should be retained. According to the embodiments of the disclosure, the epitopes with low conspicuousness are suitable for biologicals with low immune response, such as drugs or therapeutics. The epitopes with high conspicuousness scores are suitable for biologicals with high immune response, such as vaccines.

In a further aspect, the invention relates to a method of producing a vaccine composition, wherein said method comprises a step of predicting a T-cell response to an epitope. The method can be applied for predicting a T-cell response to an epitope with known epitope-TCR binding data according to the first aspect of the invention and/or for predicting a T-cell response to an epitope without a prior epitope-TCR binding data according to the second aspect of the invention.

In embodiments, the vaccine composition comprises one or more of:
at least one amino acid chain (protein, polypeptide, peptide)
at least one nucleic acid (double or single stranded RNA, DNA; DNA-RNA hybrid)
at least one immune system cell (e.g. antigen presenting cell, T-cell, B-cell, macrophage),
at least one an infectious agent (e.g. prokaryotic cell (bacteria),
at least one eukaryotic cell (yeast),
at least one virus,
at least one prion,
   and the epitope is present in the substance or vaccine as part of an amino acid sequence of the same length as the epitope or longer than the epitope, and/or as nucleic acid encoding said amino acid sequence of the same length as the epitope or longer than the epitope.

As it would be obvious to a skilled person in the art, an amino acid chain may refer to a protein, a polypeptide or a peptide.

In embodiments, nucleic acids refer to double- or single-stranded RNA, DNA and/or DNA-RNA hybrid.

Immune system cells are known in the art, the examples of immune system cells can be but are not limited to antigen-presenting cells, T-cells, B-cells, macrophages.

In embodiments, an infectious agent can be a prokaryoytic cell, such as bacteria.

In other embodiments, the vaccine composition may comprise a eukaryotic cell, such as yeast.

In another aspect, the invention relates to a vaccine composition comprising one or more epitopes with high T-cell response identified according to the methods of the present disclosure.

In another aspect, the invention relates to a non-immunogenic composition for use in a method of treating a disease comprising one or more peptides with low T-cell response identified according to the disclosed methods.

The present invention will be now described in more details, referring to examples that are not limitative.

### EXAMPLES AND/OR DESCRIPTION OF FIGURES

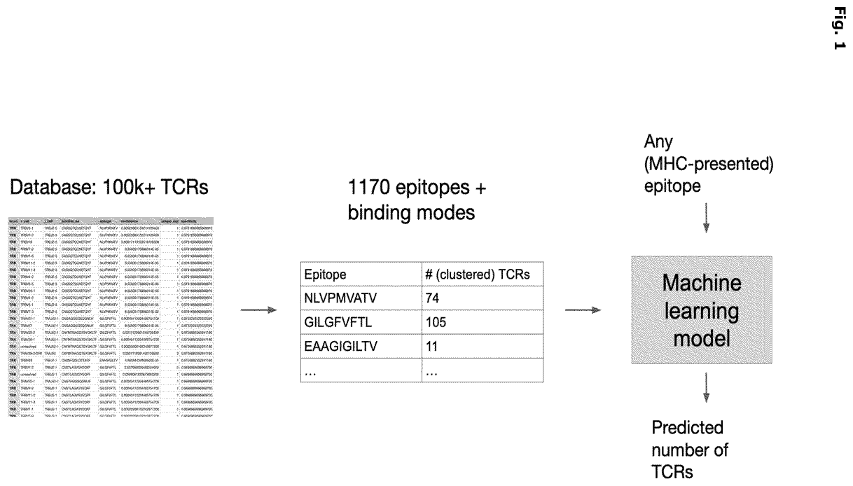

FIG. 1 workflow presents conspicuousness score calculation and training of the machine learning model.

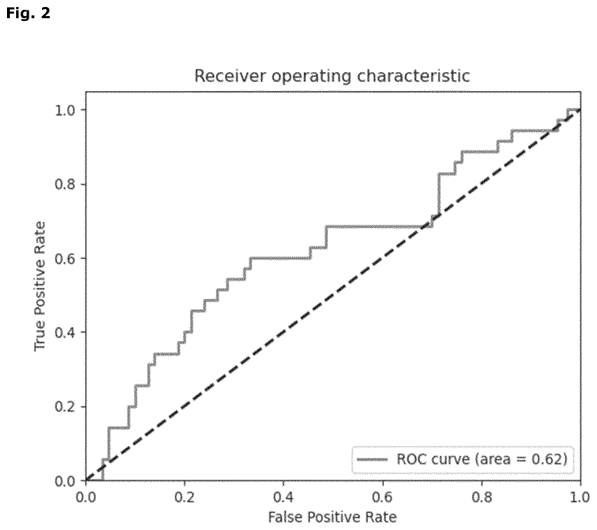

FIG. 2 shows the Receiver Operator Characteristic for the independent test data.

The present invention will now be further exemplified with reference to the following examples. The present invention is in no way limited to the given examples or to the embodiments presented in the figures.

### Example 1: Operation of the Method

An antigen amino acid sequence of a target of interest is split into epitopes based on a sliding window approach with variable lengths of 7 to 25 amino acids. Alternatively, potential epitopes of interest can be entered directly.

Each epitope in turn is given as input to the trained machine learning model that is deployed on a web platform or API or is packaged into an executable.

For each epitope, the trained machine learning model returns a conspicuousness score together with an additional interpretability measure, both representing the breadth of the T-cell response to the given epitope.

Epitopes and antigens with high scores are retained as good candidates for vaccines, while ones with low scores are retained as good candidates for biologicals.

### Example 2: Evaluation of the Algorithm for Prediction of the Conspicuousness of an Epitope

Evaluation of the machine learning models is done using 3 different types of metrics:
1. Standard regression metrics such as R-squared, RMSE and MAE due to the nature of the problem and used machine learning algorithms
2. Spearman rank correlation: the errors calculated by the regression metrics such as RMSE show how far the predicted conspicuousness score lies from the actual values. However, in the principal scenario where a list of potential candidate epitopes is given, it is more important to accurately rank them from least to most immunogenic. Therefore, the Spearman rank correlation is used to assess the algorithm's ranking ability.
3. Area Under the Curve (AUC): the problem can be transformed into a binary classification, with either low immunogenicity or high immunogenicity, for which AUC gives an additional performance indicator.

These metrics are applied using two different evaluation strategies:
1. Cross-validation: the database that is used for training is repeatedly split into training and test partitions and the aforementioned metrics are calculated and averaged. This additionally allows the selection of the best model.
2. Independent data set: an additional data set containing epitopes with known immunogenicity is used to independently verify model performance.

Results can be observed in Table 1 and FIG. 2, showing a clear signal that the algorithm has the capability to predict the conspicuousness of an epitope.

The results indicate that the present invention does not require knowledge of the TCR, as the score simply represents the size of the space and not the actual space and can be done for unseen epitopes.

TABLE 1
Evaluation metrics

| | Spearman correlation | RMSE |
|---|---|---|
| Cross-validation | 0.10 | 10.15 |
| Independent data set | 0.06 | / |

The present invention is in no way limited to the embodiments described in the examples and/or shown in the figures. On the contrary, methods according to the present invention may be realized in many different ways without departing from the scope of the invention.

1. A method of predicting a T-cell response to a query epitope with known epitope-TCR binding comprising:
calculating a conspicuousness score of said query epitope wherein, said score is calculated based on one or more of known TCR sequences responsive to said query epitope or number of TCR clusters, grouped by TCR sequence patterns, responsive to said query epitope or by a centrality metric for said query epitope in an epitope-TCR graph; and
predicting the T-cell response to the query epitope based on said calculated conspicuousness score.

2. The method according to claim 1, wherein said query epitope is present in an epitope-TCR binding pair databases.

3. The method according to claim 1, wherein said query epitopes with a conspicuousness score above a predefined threshold are selected for vaccine testing.

4. The method according to claim 1, wherein the T-cell response of said query epitopes with a conspicuousness score above a predefined threshold is measured by means of a T-cell response assay.

5. The method of claim 1, wherein said method is a computer-implemented method.

6. A computational method of predicting T-cell response to a candidate epitope comprising:
generating a training data set by calculating a conspicuousness score of a plurality of epitopes with known epitope-TCR binding wherein said conspicuousness score is calculated based on the number of known TCR sequences responsive to each of said epitope or number of TCR clusters responsive to each said epitope or by a centrality metric for each said epitopes in the epitope-TCR graph;
calculating a conspicuousness score of candidate epitope using a machine learning algorithm model trained using said training data set; and
predicting the T-cell response of candidate epitope based on said conspicuousness score.

7. The method according to claim 6, wherein said calculating conspicuousness score of candidate epitope comprises:
training the algorithm with a training data set;
feeding said algorithm with an amino acid sequence of said candidate epitope;
conversion of said sequence into numeric features wherein said numerical features represent physicochemical properties of the epitope, wherein the physicochemical properties include sequence length, HLA preference, amino acid frequencies, or molecular properties; and
predicting the conspicuousness score of the query epitope using said numeric features.

8. (canceled)

9. The method according to claim 6, wherein said method uses a predictive model, said predictive model is a machine learning model trained using a training dataset.

10. The method according to claim 6, wherein said training data set comprises a list of epitopes with known epitope-TCR binding and their conspicuousness score.

11. The method according to claim 10, wherein said list of epitopes of said training data set is derived from a database of known epitope-TCR binding pairs.

12. The method according to claim 6, wherein operation of said algorithm is based on a machine learning framework wherein said machine learning framework translates numerical input features into a single score.

13. The method according to claim 12, wherein said machine learning framework is chosen from multivariate regression, Bayesian regression, support vector machines, regression forests, linear regression, gradient descent methods, gradient boosting methods or neural networks.

14. The method according to claim 1, wherein said TCR clusters are clustered based on their TCR sequence patterns.

15. The method according to claim 1, wherein said centrality metric is chosen from degree centrality, eccentricity centrality, or PageRank centrality.

16. The method according to claim 1, wherein further transformations applied to said centrally metric to improve the quality of the metric.

17. The method according to claim 1, wherein said conspicuousness score of an epitope is used to predict T-Cell response of said epitope wherein, a low score relates to a narrow or no T-Cell response and a high score relates to a broad T-cell response.

18. The method according to claim 1, wherein said epitopes with low conspicuousness scores are suitable for biologicals with low immune response.

19. The method according to claim 1, wherein said epitopes with high conspicuousness scores are suitable for biologicals with high immune response.

20. A method of producing a vaccine composition, wherein said method comprises a step of predicting a T-cell response to a query epitope according to claim 1.

21. The method according to claim 20 wherein the vaccine composition comprises one or more of:
at least one amino acid chain,
at least one nucleic acid,
at least one immune system cell,
at least one an infectious agent,
at least one eukaryotic cell,
at least one virus, or
at least one prion,
and the epitope is present in the vaccine as part of an amino acid sequence of a same length as the epitope or longer than the epitope, and/or as nucleic acid encoding said amino acid sequence of the same length as the epitope or longer than the epitope.

22.-24. (canceled)
